(19) United States
(12) Patent Application Publication
(10) Pub. No.: US 2017/0226171 A1
(43) Pub. Date: Aug. 10, 2017

(54) ADENOCARCINOMA ANTIGENIC DETERMINANTS AND METHODS

(71) Applicant: Vaccinogen, Inc., Baltimore, MD (US)

(72) Inventors: Michael George Hanna, JR., Bethany Beach, DE (US); Jason David Howard, Woodbine, MD (US)

(73) Assignee: Vaccinogen, Inc., Baltimore, MD (US)

(21) Appl. No.: 15/090,384

(22) Filed: Apr. 4, 2016

Related U.S. Application Data
(60) Provisional application No. 62/388,858, filed on Feb. 9, 2016.

(51) Int. Cl. C07K 14/47; G01N 33/68; C07K 16/30; C07K 14/82; A61K 39/00; A61K 49/00

(52) U.S. Cl. CPC C07K 14/4748; A61K 39/0011; A61K 49/0006; C07K 16/30; C07K 14/82; G01N 33/6854; C07K 2317/21; A61K 2039/575; A61K 2039/5152; A61K 2039/55; C07K 2317/14; C07K 2317/92; C07K 2317/33; A61K 2039/57

(57) ABSTRACT

Adenocarcinoma antigenic determinants, methods for their production and use, and broadly-specific human monoclonal antibodies reactive to the epitopes. These epitopes, which can be physically recapitulated, are conserved across a range of adenocarcinomas and are capable of eliciting an immune response in humans.

### CROSS-REFERENCE TO RELATED APPLICATION

This application claims priority to U.S. Provisional Application No. 62/388,858, filed Feb. 9, 2016, the contents of which are incorporated herein by reference in its entirety.

### FIELD OF THE INVENTION

The present disclosure relates to methods of characterizing antigenic determinants which broadly define adenocarcinomas with the guidance of human monoclonal antibodies (HuMabs) reactive to such epitopes. These antibodies are generated by autologous tumor cell immunization of patients and harvesting the B-cells during a specific time period in their course of treatment. This disclosure relates in one aspect to epitopes with a high degree of inter- and intratumoral incidence across a broad range of adenocarcinomas. In another aspect, the present disclosure relates to the physical recapitulation of said epitopes in order to vaccinate humans against a wide range of adenocarcinomas using a limited number of homogeneous tumor antigens.

### BACKGROUND OF THE INVENTION

Due to the limited and inconsistent results obtained with traditional chemotherapeutic approaches, immune-based treatments of cancer are an attractive option for patient-specific therapy with the added potential for long-term surveillance and protection. However, despite positive preclinical data, the field of active specific immunotherapy (ASI) has been fraught with disappointing clinical outcomes (Kudrin and Hanna, Hum Vaccin Immunother, 8:1135-1140, 2012). This stagnation is particularly frustrating because the link between immune competence and tumor prevention has been long understood.

Neoplastic lesions are relatively common over the course of one's life and are often naturally resolved or at least inhibited by normal immune function. Based on previous autopsy studies, pathologists determined a “perfect” test for breast cancer would detect disease in at least 10% of women who die from other causes. Similar studies demonstrate that 37-57% of humans have thyroid nodules, benign lesions which can progress to invasive disease. Furthermore, 40% of men over the age of 60 and 60% of men over 80 have detectable prostate cancer cells. Yet, the rates of invasive breast, thyroid, and prostate cancer requiring treatment are much lower than these autopsy studies would suggest. Additionally, patients who lack robust immune responses, such as organ transplant recipients or long-term HIV patients, experience an increased risk of cancer development. These experiments in nature generally demonstrate an inverse correlation between immune competence and cancer development. But, the relationship between immune function and malignant disease is complex and not all responses are curative.

For example, according to the “immunoediting” hypothesis (Dunn et al., Nat Immunol, 3:991-998, 2002; Dunn et al., Annu Rev Immunol, 22:329-360, 2004; Schreiber et al., Science, 331:1565-1570, 2011), poorly immunogenic tumors which escape immune surveillance may actually be created by normal immune function through a long-term process of clonal selection. In this paradigm, immunogenic clones which arise over time are appropriately recognized by the immune system and pruned away, thus the tumor which escapes control and becomes detectable has specifically evolved to avoid competent immune recognition. The complex relationship between immune function and cancer development is undeniable. The immune system plays a dual role in cancer: it can suppress tumor growth by destroying cancer cells but also promote tumor progression by actively selecting clones which can thrive in an immunocompetent host. The latter function, while seemingly paradoxical, is an essential and mandatory component of tumor development possibly driven by antigenic competition (Hanna and Peters, J Immunol, 104:166-177, 1970). However, these inborn tools must be harnessed with the appropriate training and timing if effective preventive vaccines for cancer are to be developed. By comparison, similar efforts have completely eradicated smallpox from existence. International vaccination campaigns will soon eradicate polio. Yet, we are no better prepared to treat advanced smallpox or polio infection than our ancestors were 100 years ago. If we can learn anything from these important medical breakthroughs, we must understand the most effective vaccination strategies rely on prevention over treatment; cancer is no different.

Two basic classes of cancer vaccines currently exist: preventive and therapeutic. Thus far, preventive cancer vaccines target the few known viruses associated with cancer development. These include Gardasil® (Merck & Co) and Cervarix® (GlaxoSmithKline), which prevent high-risk human papillomavirus (HPV) infection, and Recombivax HB® (Merck & Co), which prevents hepatitis B virus (HBV) infection. These preventive measures have been highly efficacious because they adhere to the traditional dogma of vaccination: prepare the human immune system to recognize a relatively stable population of epitopes. However, the vast majority of tumors do not have a viral etiology.

Consequently, therapeutic cancer vaccines have been developed with the intent to treat established malignant disease. The clinical trials evaluating these treatments also focus disproportionately on recurrent or metastatic patients for which few options remain. Yet, recent translational research has determined that advanced tumors create very effective immunosuppressive environments, which ultimately inhibit the cytotoxicity of infiltrating lymphocytes. For example, pathways such as the PD-1/PD-L1 signaling axis are capable of converting human Th1/Th2 cells into lymphocytes which exhibit an “exhausted” phenotype. Thus, stimulating a robust immune response against target tumor antigens within this established, immunological sanctuary has proven difficult.

These results suggest efforts must be made to target cancer-specific antigens prior to the establishment of an immunosuppressive microenvironment. To this end, preclinical work with inbred guinea pigs (syngeneic hepatocellular carcinoma) (Hanna et al., Principles of Cancer Biotherapy, R. K. Oldham, Raven Press, 1987), HER2-neu transgenic mice (breast cancer) (Lollini et al., Future Oncol, 1:57-66, 2005), and other cancer models (Shirota and Klinman, Immunotherapy, 5:787-789, 2013) have demonstrated cancer vaccination strategies decrease in efficacy as a function of advancing disease. For example, in the aforementioned guinea pig model, the adaptive immune system was capable of eliminating tumor metastases 0.1-0.2 mm in size with at least 75% efficacy. By comparison, tumors 0.35-0.5 mm in diameter could not be treated with any degree of success (Hanna et al., 1987). The implications of these results are striking. Both tumor sizes are below the current limit of detection for diagnostic imaging technology. Modern clinical PET scanners have a 4 mm limit of resolution. Therefore, while the difference between immunologically treatable (0.1-0.2 mm) and clinically detectable (4 mm) may seem modest with respect to diameter, this linear discrepancy corresponds to an 8,000- to 64,000-fold difference in spherical volume. As the totality of cells within a burgeoning tumor increases, enhanced cellular diversity and immunological barriers become more problematic. By way of analogy, this is the equivalent of treating every infectious disease patient only after sepsis has developed. Positive outcomes are possible, but the odds of success have been greatly reduced.

Based on the accumulated knowledge of animal models and human clinical observations, a normal immune system has the capability to prevent or inhibit malignant growth. The existence of molecular determinants which differentiate between normal tissue and invasive tumors allows for this possibility. However, they must be exploited at the appropriate time. Markers of neoplastic disease must be targeted after cellular transformation but prior to the establishment of an immunosuppressive microenvironment. On the other hand, by definition, these determinants are not ideal immunological targets. Problematic malignancies evolve a repertoire of mutant proteins or novel protein-protein interactions which are biologically dysfunctional, but not strongly immunogenic. This effectively creates an immunological “blind spot” to allow tumor development to progress with impunity.

However, post-surgical, patient-specific active immunotherapy (autologous tumor vaccine treatment) has demonstrated an ability to effectively program the adaptive immune system to recognize these blind spots, destroy recurring malignancies (Vermorken et al., Lancet, 353:345-350, 1999), and establish long-term protection (de Weger et al., Clin Cancer Res, 18: 882-889, 2012). Yet, even this approach has only been effective in the setting of minimal residual disease, most likely for the reasons outlined above. These options must be deployed prior to the establishment of an effective immunosuppressive microenvironment (stage II vs. stage III colon cancer) (Vermorken et al., 1999; Hanna et al., Hum Vaccin, 2:185-191, 2006). Thus, it follows the earliest possible interventions have the best opportunity to prevent cancer-related death.

What is needed are compositions and methods for appropriately utilizing immune-based products provided by patient-specific, active immunotherapy to obviate the need for advanced cancer treatment. In other words, the immune system must be allowed to identify molecular determinants which can be used to recapitulate effective immunogens for the prevention of malignant disease. Through ASI, these humoral tools are created by agnostic immune systems to affect immediate clinical benefit and establish long-term immune surveillance. While ASI has proven that antigenic heterogeneity and early intervention is required for effective cell-mediated cancer treatment, the humoral homogeneity uncovered by these methods and described with greater detail herein may be sufficient for general cancer prevention.

### BRIEF SUMMARY OF THE INVENTION

The present disclosure is based, in part, on methods for characterizing and producing specific epitopes common to a broad range of adenocarcinomas. These epitopes are identified by and are reactive with specific HuMabs (ASI-HuMabs) also produced according to methods disclosed herein. Such antibodies are substantially non-reactive with normal tissue. Moreover, the epitopes defined by the antibodies are capable of eliciting an immune response in humans against adenocarcinomas.

These methods involve harvesting B-cells within a specific window of opportunity from humans immunized with patient-specific, ASI (i.e. autologous, live, tumor cell vaccines). The isolated HuMabs are screened and selected to obtain immunoglobulins which demonstrate broad inter- and intratumoral reactivity, as measured by direct immunohistochemistry, while being substantially non-reactive with normal tissues.

The present disclosure further provides methods for using these ASI-HuMabs as molecular probes to identify and characterize the amino acid sequence of specific epitopes within a subclass of tumor antigens, i.e., antigens that are homogeneously expressed within, in this case, adenocarcinomas. In another aspect, the present disclosure provides methods for preparing specific peptides or producing recombinant proteins from the base sequence of the identified epitopes.

In yet a further aspect, the present disclosure provides methods for screening and selecting the synthesized or recombinant proteins for immunogenic potential and identifying those capable of eliciting an appropriate immune response. In a particular aspect, the epitopes are selected by in vitro T-cell blastogenesis and/or in vivo delayed-type hypersensitivity (DTH) reactions in patients immunized with autologous tumors and, in one aspect, include a plurality of epitopes selected from the protein subunits of CTAA 28A32 (Ransom et al, Int J Cancer, 54:734-740, 1993), an antigenic complex recognized by ASI-HuMab MCA 28A32. In another aspect, a plurality of such epitopes from a collection of antigens identified by additional ASI-HuMabs is combined for a multi-valent preparation and the molar ratios of the components are adjusted to limit antigenic competition and attain optimal human immunogenic activities in pharmacological compositions.

While not wishing to be bound by theory, we believe the novel use of ASI-HuMabs as described herein, specifically HuMabs screened from B-cells of cancer patients having previously demonstrated immunoprotection against residual occult disease (Vermorken et al., 1999), is the only successful approach for obtaining and recapitulating epitopes capable of eliciting an immunogenic response to a wide range of emerging adenocarcinoma subtypes. These antigenic determinants can be optimally utilized for prophylaxis prior to the establishment of an immunosuppressive tumor microenvironment or any other form of systemic immune suppression.

These and other objects, features, aspects, and advantages of the present patent document will become better understood with reference to the following description and accompanying drawings.

### BRIEF DESCRIPTION OF THE DRAWINGS

FIG. 1. Distribution of antibody reactivity as determined by positive indirect immunoperoxidase staining. Ten ASI-HuMabs were screened against 15 patient-derived colorectal tumor sections. All tumors were moderately well- to well-differentiated, with the exception of Patient 9 (poorly differentiated). All tumors were primary malignancies, except Patient 4 (liver metastases). (Adapted from Haspel et al., Cancer Res, 45:3951-3961, 1985)

FIG. 2. Two ASI-HuMabs react with most colorectal tumors in a selected cohort. Positive indirect immunoperoxidase staining of two ASI-HuMabs against 15 colorectal paraffin sections (left column) and 9 air-dried Cytospin preparations of dissociated tumors (right column). A limited and manageable number of ASI-HuMabs may be capable of elucidating the antigenic determinants for a large number of adenocarcinomas. (Adapted from Haspel et al., 1985)

FIG. 3. Schematic workflow associated with ASI-HuMab production and investigation. Patients are inoculated with their own sterile, metabolically-active, but non-tumorigenic cancer cells admixed with TICE-BCG. Subsequent vaccinations consisting only of tumor cells serve to further boost immunity against minimal residual disease and provide evidence (with DTH reactions) of appropriate immunological recognition. B-cells isolated from vaccinated patients are harvested and screened for antibodies which demonstrate broad inter- and intratumoral reactivity with adenocarcinomas while exhibiting little to no reactivity against an array of normal tissues (ASI-HuMabs). These antibodies are then used to characterize their associated antigenic determinants (epitopes) which can be physically recapitulated and tested for humoral and cell-mediated specificity and reactivity.

Table 1. 88BV59 Reactivity (≧2+) with human tumor samples. Biotinylated 88BV59 reactivity was screened against a wide variety of primary human malignancies using a conventional avidin peroxidase detection system. Various concentrations of antibody were used to demonstrate the affinity and specificity of staining. All slides were scored 0-4+ by a trained pathologist. These data simultaneously demonstrate the broad (intertumoral) and specific (adenocarcinoma) nature of 88BV59 immunoreactivity within a wide range of tumor samples. This suggests antigenic homogeneity exists across tumors with shared cellular origin.

Table 2. Reactivity of 88BV59 with normal human tissue. Biotinylated 88BV59 reactivity was screened against a wide variety of normal human tissues using a conventional avidin peroxidase detection system. Representative concentrations of 88BV59 utilized in this screen were sufficient to positively stain the majority of carcinomas arising from simple epithelium. The low degree of reactivity against normal tissue demonstrates the cancer specificity of 88BV59. The vast majority of normal tissue reactivity is characterized as focal or non-specific (see pathology notes).

FIG. 4. Genomic and transcriptional alterations of KRT8 in various human cancers. FIG. 4A: The alteration frequency of KRT8 in cancer mutation profiling databases (The Cancer Genome Atlas, TCGA). Alterations are considered any mutation (green), deletion (blue), amplification (red), or combination of these dysfunctions (grey) in >21,000 tumors of various subtypes. Conserved genetic or transcriptional alterations of KRT8 are not frequent enough to explain the broad reactivity which 88BV59 demonstrates against glandular carcinomas. FIG. 4B: A one-dimensional representation of KRT8 mutations observed in the investigations above. The antigenic region responsible for 88BV59 binding is also depicted. Images were created in cBioPortal for Cancer Genomics.

FIG. 5. Genomic and transcriptional alterations of ENO1 in various human cancers. FIG. 5A: The alteration frequency of ENO1 in cancer profiling databases (TCGA). Alterations are considered any mutation (green), deletion (blue), amplification (red), or combination of these dysfunctions (grey) in >21,000 tumors or various subtypes. Conserved genetic or transcriptional alterations of ENO1 are not frequent enough to explain the broad reactivity observed between MCA 28A32 and carcinomas. FIG. 5B. A one-dimensional representation of ENO1 mutations observed in the investigations above. Images were created in cBioPortal for Cancer Genomics.

Table 3. Partial protein sequence analysis of CTAA 28A32-32K. Purified CTAA 28A32-32K was cleaved with cyanogen bromide and the resulting peptide fragments were identified using a gas-phase protein sequencer. Despite significant sequence variations compared to most annexins, the amino acids analyzed share 74% identity with annexin A4 (ANXA4). (Adapted from Pomato et al., Vaccine Research, 3:145-161, 1994)

FIG. 6. Genomic and transcriptional alterations of ANXA4 in various human cancers. FIG. 6A: The alteration frequency of ANXA4 in cancer profiling databases (TCGA). Alterations are considered any mutation (green), deletion (blue), amplification (red), or combination of these dysfunctions (grey) in >21,000 tumors or various subtypes. Conserved genetic or transcriptional alterations of ANXA4 are not frequent enough to explain the broad reactivity observed between MCA 28A32 and carcinomas. FIG. 5B. A one-dimensional representation of ANXA4 mutations observed in the investigations above. Images were created in cBioPortal for Cancer Genomics.

FIG. 7. ASI-HuMab colon cancer cross-reactivity. Dissociated colon cancer preparations from OncoVAX® patients (n=49) were screened against 9 ASI-HuMabs for immunohistochemical reactivity. The percentage of tumor cells from each sample which reacted with a given ASI-HuMab was quantified with a cytometer. Degree of reactivity was color-coded according to the legend below. A white box indicates the reaction was not performed. KRT18 and CEA are common histochemical positive controls for colon adenocarcinomas.

FIG. 8. ASI-HuMab rectal cancer cross-reactivity. Dissociated rectal cancer preparations from OncoVAX® patients (n=6) were screened against 9 ASI-HuMabs for immunohistochemical reactivity. The percentage of tumor cells from each sample which reacted with a given ASI-HuMab was quantified with a cytometer. Degree of reactivity was color-coded according to the legend. A white box indicates the screen was not performed. KRT18 and CEA are provided as common histochemical positive controls for gastrointestinal adenocarcinomas.

FIG. 9. ASI-HuMab colon and rectal inter- and intratumoral cross-reactivity. Based on the ASI-HuMab heat maps, the degree of inter- and intratumoral cross-reactivity could be determined for each antibody within colorectal cancers. FIG. 9A,B: Any degree of immunohistochemical staining by a given ASI-HuMab was considered positive tumor recognition. The percentage of tumors recognized within a given cohort was considered the intertumoral reactivity of the ASI-HuMab or histochemical marker. FIG. 9C,D: The percentage of cells within each tumor recognized by a given ASI-HuMab was averaged across the entire cohort. The resulting mean was considered the average intratumoral reactivity of the given ASI-HuMab or histochemical marker.

Table 4. Comparison of antigen expression by immunohistochemical analysis with DTH response. ^{a} Mean diameter of erythema (mm). No response (−). ^{b} Staining intensity: (−) negative to very weak focal staining (<5%); 1+, weak staining; 2+, weak to moderate staining; 3+, moderate staining; 4+, strong staining (Adapted from Bloemena et al., Cancer Res, 53:456-459, 1993).

Table 5. Proliferative responses to tumor-associated antigens by peripheral blood lymphocytes from patients immunized with an autologous tumor-cell BCG vaccine. Although peripheral blood lymphocytes demonstrate little reactivity towards CTAA 16.88 or 28A32 prior to vaccination, specific reactivity increases against CTAA 28A32-32K with increasing vaccination. ¹ SI (given in parentheses)≧3.0 were considered positive. ² Time relative to vaccination. Baseline is before vaccination but after surgery. The 3^{rd} and 4^{th} vaccinations were delivered 2 weeks and 6 months after the first vaccination, respectively (Adapted from Ransom et al., 1993).

### DETAILED DESCRIPTION OF THE EMBODIMENTS

While not wishing to be bound by theory, we believe two significant issues were major hindrances for effective cancer vaccine development. The first occurred with the false assumption that tumors share a relative level of genetic homogeneity. Within this philosophy, allogeneic tools could be built from “off-the-shelf” antigens identified by genetic screening, which would be able to treat a large number of patients. Unfortunately, the resulting vaccines failed to provide significant clinical efficacy, especially in advanced patients. The second misconception occurred once the virtually unlimited nature of tumor genetic diversity was truly understood with efficient DNA sequencing methods (Vogelstein et al., Science, 339:1546-1558, 2013; Wood et al., Science, 318:1108-1113, 2007; Ling et al., PNAS, 112:E6496-6505, 2015). It was discovered that thousands of genes causally linked with the development of cancer demonstrate a unique spectrum of dysfunction and distribution leading to near limitless inter- and intratumoral heterogeneity. Based on these results, clinicians and researchers understandably presumed this nearly infinite inter- and intratumoral genetic heterogeneity would also be represented on the immunologic level, providing a serious limitation to general cancer vaccine development. It followed that no one set of predefined genetic targets could ever be representative of a meaningful number of patients.

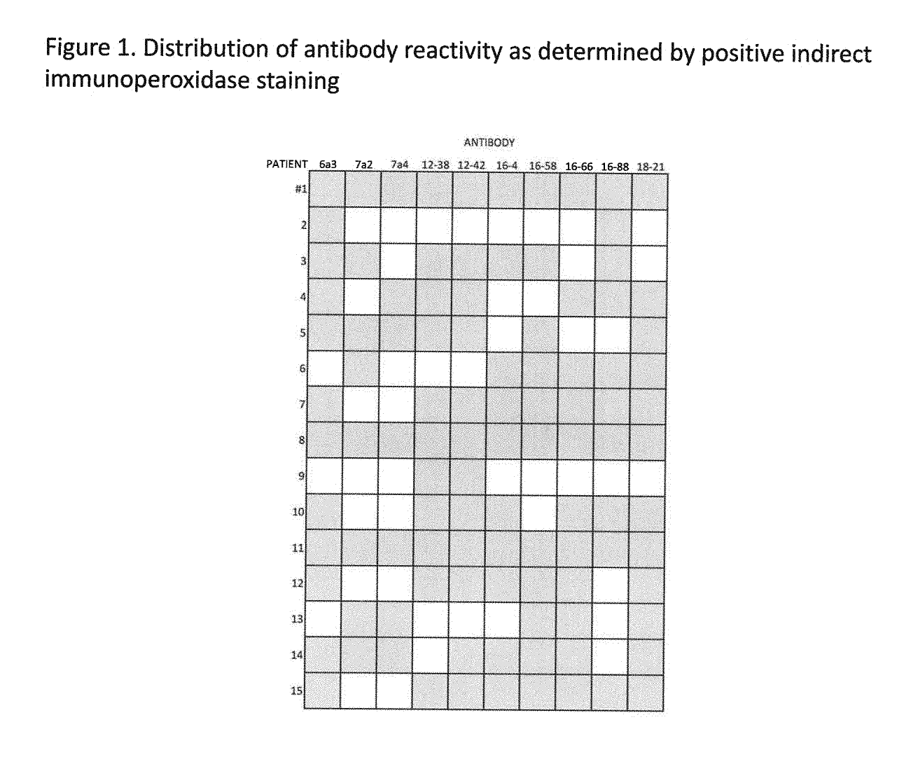

While the genomic heterogeneity of cancer is undeniable, based on the present methods and compositions, a previously unappreciated level of homogeneity exists on the immunologic level (Table 1, FIGS. 1,2, 7-9). Aspects of the present methods are based in part on the immune system targeting these homogeneous antigens before a given tumor has the ability to create an immunosuppressive microenvironment. In fact, the data suggests a manageable number of antigenic determinants (5-10 epitopes) could broadly characterize a surprisingly large number of tumors. These conclusions can now be made based on the greater breadth of cross-reactivity data provided herein (Tables 1 and 2, FIGS. 7-9), a greater understanding of the precise cross-reactivity pattern of these tools (i.e. adenocarcinoma-specific), and modern advances in peptide synthesis and recombinant protein expression. Faced with incomplete cross-reactivity data and limited technology, these earlier studies could not have anticipated an entire subclass of tumors could be defined immunologically with a limited set of antigenic determinants which could then be physically recapitulated for general vaccine development. With the benefit of modern technology and insight, these are the unique, pivotal, and constructive observations which will lead to the development of a prophylactic adenocarcinoma vaccine.

TABLE 1
88BV59 Reactivity (≧2+) with human tumor samples.

| | 88BV59 (μg/ml) | | | | | | | |
|---|---|---|---|---|---|---|---|---|
| Tumor Type | 1.25 | | 0.625 | | 0.313 | | 0.156 | |
| Colon | 96/99 | (97%)¹ | 94/99 | (95%) | 87/98 | (89%) | 63/76 | (83%) |
| Breast | 16/16 | (100%) | 15/16 | (94%) | 14/16 | (88%) | 10/15 | (66%) |
| Stomach | 8/8 | (100%) | 3/3 | (100%) | 3/3 | (100%) | 3/3 | (100%) |
| (Gastric Adenocarcinoma) | | | | | | | | |
| Prostate | 6/6 | (100%) | 6/6 | (100%) | 6/6 | (100%) | 3/3 | (100%) |
| Lung Adeno | 7/7 | (100%) | 7/7 | (100%) | 7/7 | (100%) | 5/6 | (80%) |
| Lung Squamous | | | | | | | | |
| Poorly differentiated | 7/7 | (100%) | 7/7 | (100%) | 3/7 | (43%) | 2/7 | (28%) |
| Mod-well differentiated | 0/3 | (0%) | 0/3 | (0%) | 0/3 | (0%) | 0/3 | (0%) |
| Ovarian | 10/10 | (100%) | 10/10 | (100%) | 9/9 | (100%) | 5/7 | (71%) |
| Melanoma | 0/2 | (0%) | 0/2 | (0%) | 0/2 | (0%) | 0/2 | (0%) |
| Lymphoma | 0/3 | (0%) | 0/3 | (0%) | 0/3 | (0%) | 0/2 | (0%) |
| Head and Neck (squamous) | 0/4 | (0%) | 0/4 | (0%) | 0/4 | (0%) | 0/4 | (0%) |
| lymphoma, melanoma | | | | | | | | |

Reactivity is defined as staining intensity of ≧2+. Reactivity is determined on a 0-4+ scale with 0 = negative; 1+ = weak; 2+ = moderate; 3+ = strong; and 4+ = very strong.
¹Number of reactive/total (percent reactive)

Previous methods of identifying tumor-specific antigens have been insufficient, at least in part, because they involve an “inside-out” approach. Traditional genetic profiling studies begin with DNA- or RNA-based targets (“inside”) and assume these results are significantly represented within the fully translated proteome (“outside”). Thus, cancer-specific protein-protein interactions, changes in subcellular localization, and a variety of post-translational modifications are completely absent from the potential pool of clinical targets.

An improved method for identifying immunogenic tumor-specific antigens is based on an “outside-in” approach. If we intend to employ the immune system to treat cancer, we must adopt its perspective and not assume a list of components (“genome”) adequately describes the myriad functional activities of a living cell (“proteome”, “interactome”, “immunome”, etc). Any meaningful understanding of immune interaction with human tissue should start from the immune system's perspective in order to capture the full diversity of potential antibody targets. Consequently, the preferred methods, as described herein, uniquely exploit the human immune system to determine which targets within the proteomic milieu are simultaneously cancer-specific and immunologically relevant. By definition, because this vaccination process is autologous, antigens defined as “self” are inherently ignored and disease-specific mutations, protein-protein interactions, and post-translational modifications become the immediate immunological focus. This “outside-in” approach is a preferred method for creating an agnostic molecular database of cancer-specific immunogens.

Adopting the perspective of the immune system for future clinical benefit is critical because normal immune function is a potential mechanism for creating the surprising level of antigenic homogeneity observed with this method (Table 1, FIGS. 1, 2, 7-9). In a process termed “immunoediting”, it is thought that neoplastic cells commonly arise and are resolved with regularity (Dunn et al., 2002; Dunn et al., 2004; Schreiber et al., 2011). However, in some instances, these cells can cycle over time through a process of immune selection. During this time, highly immunogenic clones are recognized and destroyed while poorly immunogenic clones become a larger and larger proportion of the burgeoning collective. Supporting this theory is the observation that immune compromised animals create tumors with enhanced immunogenicity compared to fully-competent animals (Shankaran et al., Nature, 410:1107-1111, 2001). Because immunoediting is not involved with the tumor evolutionary process in these animals, the resulting cells display neoantigens more reactive within normal functioning immune systems.

Thus, the tumor which ultimately escapes immune control and becomes detectable is actually honed and crafted by normal immune function to be poorly immunogenic. This more recent model corresponds well with previous studies of antigenic competition (Hanna and Peters, 1970). During the process of immunoediting, primary focus on highly immunogenic clones may force weaker, oncogenic antigens to be unrecognized and preserved over time. With this in mind, it may be more accurate to classify tumor creation as a long-term active dysfunction of immune response, a form of immunotolerance, rather than simply the absence of immune surveillance. While these hypotheses are critically important for understanding the life cycle of malignant disease, we believe this model also provides an attractive explanation for tumor immunological homogeneity: the healthy immune system imposes a homogeneous requirement upon developing tumors, forcing them into “immunological blind spots”. This mechanism of tumor evolution may provide a unique clinical opportunity as these blind spots could be inter- and intratumoral requirements which can be anticipated, predicted, and exploited on a molecular level. In this paradigm, general vaccine development with strong antigens is not required as these epitopes are sufficiently recognized by normal immune function. What is needed are means for heightening the recognition of relatively weak, cancer-specific, homogeneous antigens.

With this in mind, we have found that humans can be inoculated with their own tumor cells, which have been specially processed to destroy the immunosuppressive microenvironment, preserve immunogenicity, and inhibit tumorigenic potential. These tumor cells, when combined with an appropriate adjuvant, generate cancer-specific HuMabs (ASI-HuMabs) after intradermal injection. Because the vast majority of proteins expressed by these cells have previously been defined as “self”, typical human proteins, patient-specific splice variants, and single nucleotide polymorphisms (SNPs), for example, do not stimulate a humoral response or molecular memory. This in-born background correction greatly increases the proportion of cancer-specific B-cells in peripheral lymphocytes post-vaccination (Haspel et al., 1985). Antibodies produced by these B-cells from autologous human vaccination are then harvested and characterized as described herein for broad tumor specificity by, for example, flow cytometric or immunohistochemical analyses.

These antibodies play a critical role in cancer detection and destruction. Previous studies in HER2-neu transgenic mice demonstrated immunoprotection against early disease depends greatly on the ability to make antibodies (Nanni, et al., J Immunol, 173:2288-2296, 2004). B-cell deficient HER2-neu mice pretreated immunologically do not receive the same level of tumor protection as antibody-competent counterparts. Furthermore, this protective effect is specifically associated with IgG2a and -2b antibodies, which are associated with T-helper cell responses induced by IFN-γ. More recent work has demonstrated that pregnant, vaccinated HER2-neu mice are capable of transmitting cancer-protective antibodies to their young via lactation (Barutello, et al., Oncoimmunology, 4:e1005500, 2015). Clearly, humoral identification of malignant cells in animals and patients not only mediates clinically meaningful responses, but provides a method for identifying disease-relevant antigenic determinants for additional clinical benefit.

Thus, in one aspect, the methods described herein rely on ASI, a treatment paradigm which uses a patient's own tumor to boost immune responses against residual cancer cells capable of reemerging within the patient's lifetime. This approach is in contrast to passive immunotherapy, which utilizes systemically reactive products to encourage general antitumor responses. Previous studies evaluating ASI in colon cancer patients have determined significant efficacy and minimal toxicity is associated with this treatment (Vermorken et al., 1999; Hanna et al., 2006). Therefore, by definition, the antigenic determinants informed by ASI-HuMabs have been associated with prior positive clinical outcome, rather than assuming a given mutation is adequately expressed and simultaneously immunogenic. Additionally, these tumor antigens are either absent or quantitatively decreased on normal cells, a key difference the adaptive immune system is able to recognize and exploit. Moreover, we have found a surprising degree of cross-reactivity with ASI-HuMabs which can recognize a broad range of adenocarcinomas due to their shared cell of origin (i.e. simple epithelium).

Thus, according to one aspect of the methods described herein, once a patient's immunological memory has been established post-vaccination, these patients are screened for the production of B-cells which produce cancer-specific antibodies. More specifically, following adjuvanted inoculation with a patient's own tumor, B-cells are isolated from peripheral blood lymphocytes and the resulting ASI-HuMabs produced therein are screened against tumor samples from a broad range of cancers. Such antibodies which demonstrate broad inter- and intratumoral adenocarcinoma reactivity are also screened against corresponding normal tissue to determine the cancer-specificity and potential clinical utility of each ASI-HuMab. Generating and screening such antibodies may be conducted as described in U.S. Pat. Nos. 5,348,880; 5,521,285; and 5,951,985, each of which is incorporated by reference herein. However, these prior methods focused on utilizing the resulting ASI-HuMabs for cancer diagnosis and treatment because the knowledge base and technology did not exist to disclose or suggest such antibodies might demonstrate broad adenocarcinoma homogenous reactivity which could reasonably be recapitulated on the molecular level for potentially preventive benefit.

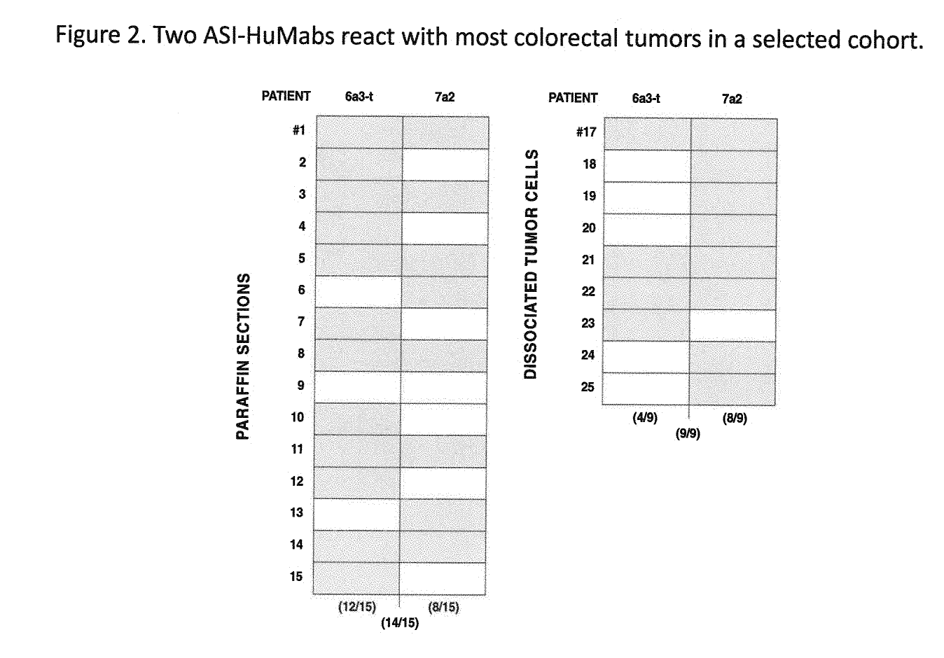

Based on our broader investigations described herein and coupled with significant advances in DNA sequencing, molecular biology, protein synthesis, and monoclonal antibody production, novel characteristics and uses for such antibodies are now provided which could not and were not previously envisioned. In fact, we have identified at least 18 ASI-HuMabs which react with a surprising degree of homogeneity across two cohorts of human colon tumors and a small cohort of rectal tumors (FIG. 1, FIGS. 7-9). Two of these HuMabs (6a3-1 and 7a2) cumulatively react with 96% of the first colon tumor cohort (FIG. 2). An additional two HuMabs (16.88 and 28A32) not only demonstrate a high level of colon intertumoral recognition (100%), but also excellent intratumoral reactivity (FIGS. 7-9). Another antibody created with the same autologous treatment method (88BV59) reacts with a majority of adenocarcinomas (i.e. breast, prostate, etc.), but not with tumors which arise from tissues other than simple epithelium (i.e. melanoma, lymphoma, etc.)(Table 1). This antibody also does not significantly react with normal human tissue (Table 2). During the course of these initial investigations, the full context of tumor heterogeneity as we know it today was not known. To be fair, the immunological homogeneity uncovered by these antibodies was appreciated at the time for an opportunity to create widely-applicable, targeted cancer therapeutics or diagnostics (Pomato et al., 1994; Wolff et al., Dis Colon Rectum, 41:953-962, 1998; Serafini et al., J Clin Oncol, 16:1777-1787, 1998). However, they were unappreciated as a means for directly capitalizing on a clinical level from the molecular targets these remarkable tools were discovering. With the benefit of modern protein synthesis, expression, and manufacturing, these antibodies can now be leveraged to their fullest extent. These and future HuMabs isolated from additional OncoVAX® patients in an upcoming phase III trial will be evaluated in this manner.

TABLE 2
Reactivity of 88BV59 with normal human tissue

| | Number | 88BV59 (μg/ml) | | |
|---|---|---|---|---|
| Tissue | Tested | 0.313 | 0.156 | Pathology Comments |
| Adrenal | 3 | 0¹ | 0 | |
| Bladder | 3 | 2 (66%)² | 1 (33%) | Focal staining limited to areas of urothelium. |
| Bone marrow | 3 | 0 | 0 | |
| Brain | 5 | 1 (20%) | 0 | Glial cells/astrocytes stained. Strongest near meninges. |
| Breast | 7 | 7 (100%) | 6 (85%) | Stains ~50% of ductal tissue. |
| Cervix | 3 | 3 (100%) | 3 (100%) | Focal staining of endocervix only; exocervix not stained. |
| Colon | 14 | 10 (71%) | 9 (64%) | Focal staining limited primarily to brush border. |
| Esophagus | 3 | 1 (33%) | 1 (33%) | Glands only in one tissue. Squamous epithelial lining was |
| | | | | non-reactive. |
| Eye (Optic Nerve) | 3 | 0 | 0 | Does stain lacrimal gland 3-4⁺. |
| Heart | 6 | 0 | 0 | |
| Kidney | 7 | 6 (85%) | 3 (42%) | A portion (<50%) of collecting tubules stained; glomeruli |
| | | | | not stained. |
| Lymph node³ | 9 | 0 | 0 | (3) Includes non-involved lymph nodes from tumor patients. |
| Liver | 9 | 0 | 0 | Only bile ducts in portal tract were stained. Parenchyma |
| | | | | negative. |
| Lung | 3 | 0 | 0 | |
| Muscle | 4 | 0 | 0 | |
| Ovary | 10 | 0 | 0 | |
| Pancreas | 5 | 2 (40%) | 1 (20%) | Stains ducts and arterioles only; acinii are negative. |
| Parathyroid | 4 | 0 | 0 | |
| Pituitary | 3 | 3 (100%) | 1 (33%) | Focal staining of epithelial cells. |
| Prostate | 6 | 5 (83%) | 1 (17%) | Stains ductal epithelial tissue only. |
| Salivary gland | 6 | 6 (100%) | 6 (100%) | Only secretory ducts were stained. |
| Skin | 5 | 2 (40%) | 1 (20%) | Stains ducts in dermis only; 3 samples do not have dermal |
| | | | | layer. Epidermis was non-reactive |
| Small intestine | 3 | 3 (100%) | 3 (100%) | Focal staining of epithelium primarily limited to brush |
| | | | | border. |
| Spinal cord | 4 | 4 (100%) | 0 | Primarily while matter stains. |
| Spleen | 4 | 0 | 0 | |
| Stomach | 4 | 4 (100%) | 4 (100%) | Mainly stains edge of section; deep glands were weak to |
| | | | | negative. |
| Testes | 5 | 0 | 0 | |
| Thymus | 5 | 3 (60%) | 0 | Weak focal reactivity with epithelial lining of thymus. |
| Thyroid | 6 | 0 | 0 | |
| Tonsil | 6 | 6 (100%) | 6 (100%) | Very focal (<5% of section) reactivity with epithelial |
| | | | | framework of follicles. |
| Uterus | 5 | 4 (80%) | 3 (60%) | Staining limited to glands within the endometrium. |

Reactivity is defined as ≧2+ at 0.156 μg/ml, the concentration 88BV59 stained 80% of colon tumors tested.
¹Number of samples with ≧2+ reactivity.
²( ) = percentage of samples with ≧2+ reactivity.

In one aspect, the present disclosure relates to using such ASI-HuMabs for the production of synthetic peptides or recombinant proteins to elicit an immune response against the emergence of a broad range of adenocarcinomas. By “immune response” herein, we mean that the synthesized peptide, recombinant protein, or a combination of such molecular entities, when administered via intradermal injection is capable of eliciting a humoral and cell-based cytotoxic adaptive immune response. These antigens could be delivered as a collection of free peptides, antigenic complexes, or fully-expressed and presented to the immune system within a transduced allogeneic cell line. Due to the shared nature of antigens discovered in this manner, coupled with the long-term effectiveness of OncoVAX® treatment (15-year follow-up) (deWeger et al., 2012), we believe these antigenic determinants serve as a significant cornerstone of this remarkable clinical benefit (Bloemena et al., 1993; Ransom et al., 1993). Recurrence of disease in immunized patients expressing these common antigens may also serve as periodic boosters strengthening this long-term immunological memory.

Utilizing the broadly-specific ASI-HuMabs as identified and described herein, the shared epitopes define the malignant immunome of a population of tumors, thus providing immunogenic and T-cell reactive epitopes common to a variety of different adenocarcinomas. By “broadly-specific” herein, we mean an antibody reactive with a broad range of adenocarcinomas (i.e. breast, colon, prostate, etc.) while simultaneously having little to no reactivity with normal tissue.

In one aspect, the present methods generate cancer-specific antibodies with broad adenocarcinoma reactivity. In one case, such broadly-specific ASI-HuMabs are created by inoculating humans with their own colon tumors adjuvanted with an appropriate immunostimulant, including but not limited to, Bacillus Calmette-Guerin (BCG). The initial clinical intent of OncoVAX® technology (Vaccinogen, Inc.) was to train the immune system to destroy minimal residual disease and prevent post-surgical disease recurrence. This treatment strategy was ultimately successful in stage II colon cancer patients, reducing the risk of recurrence from 1 in 3 patients to 1 in 10 (Vermorken et al., 1999). However, we have found that such methods for autologous immunization can also be used to generate B-cells which produce HuMabs with adenocarcinoma-specific cross-reactivity. It was recently determined that at least 18 of these antibodies react with colon tumors (subtype reactivity) and at least one of these antibodies can specifically recognize adenocarcinomas arising from simple epithelium (subclass reactivity) with nominal recognition of normal tissue. Due to the relative specificity of such human antibodies, the method also extends to identifying a panel of epitopes broadly defined by their shared cellular origin which could not have previously been manufactured on a meaningful scale. Using modern cell culture and screening technologies, additional cancer patients will be inoculated with their own tumors to create a larger pool of ASI-HuMabs in order to fully elucidate the entire repertoire of antigens associated with malignant carcinomas.

Accordingly, in another aspect, the resulting ASI-HuMabs are then used in techniques designed to reveal their immunogenic, adenocarcinoma-specific epitopes, which are then physically recapitulated to produce immunogenic compositions. Rather than target a handful of exogenous infectious agents potentially associated with future cancer development, these compositions could precondition the body to recognize the endogenous epitopes associated with an emerging carcinoma, which otherwise would become invasive. Moreover, in one aspect, compositions containing these shared epitopes have the ability to target the emerging carcinoma cells and deliver a potent immune response prior to the establishment of an immunosuppressive environment.

Once ASI-HuMabs have been identified with broad inter- and intratumoral adenocarcinoma reactivity and minimal normal tissue recognition, they are used to immunoprecipitate their specific target or complex of proteins from cancerous source material. This source material can be comprised of tumor cell lines, human xenografts, or human surgical resections. Using standard molecular biology techniques including by not limited to high pressure liquid chromatography (HPLC), mass spectrometry-based sequencing, hydrogen/deuterium exchange (H/DX) protein mass spectrometry, X-ray crystallography, protein microarrays, tissue microarrays, etc., the proteins associated with the antigen of interest are identified and fully characterized. Once the antibody-specific epitopes have been mapped, these peptide sequences are physically recapitulated using standard molecular biology methods, including but not limited to, liquid-phase peptide synthesis, solid-phase peptide synthesis, or recombinant protein expression (i.e. bacterial, yeast, mammalian cell lines). Linear epitopes and cancer-specific protein sequences are highly amenable to these methods. However, if these cancer-specific linear epitopes existed in abundance, they would likely be represented on the genetic level. Thus, cancer-specific conformational epitopes may require higher order protein structure and peptide engineering to elicit the same humoral and cell-mediated responses. Fusion proteins of various peptides or unique recombinant proteins may also be created or expressed to recapitulate the required three dimensional structure for appropriate antigen simulation and immune recognition of cancer-specific protein complexes.

After successful antigen recapitulation, B-cell and T-cell reactivity and/or DTH responses from immunized patients can be investigated in vitro or in vivo to determine which peptides are the most broadly-specific and capable of eliciting an immune or, in other aspects, a T-cell cytotoxic response (Bloemena et al., 1993).

Methods for screening and selecting the epitopes for desired immunogenic potential include conducting in vitro T-cell blastogenesis and/or in vivo DTH evaluation in autologous tumor immunized patients. The recapitulated antigens are thus selected for those epitopes demonstrating immunogenic potential and those antigens capable of eliciting an immune response. In a particular aspect, the antigenic determinants are selected from the group consisting of the plurality of subunits which constitute CTAA 28A32.

In yet another aspect, formulations containing a plurality of such recapitulated epitopes identified by additional ASI-HuMabs, with or without an adjuvant, are formulated to provide compositions for use in vaccination programs, by induction and/or booster immunizations, for eliciting an immune response to adenocarcinoma antigenic determinants. Molar ratios of the peptides may be adjusted to achieve desired human immune activities and limit antigenic competition.

While not wishing to be bound by theory, based on the data from previous investigations, it is unlikely that one or two antigens will be sufficient to define a broad range of tumors; however, five to ten antigens could adequately represent up to 60% of cancer subtypes, as the most common cancers (adenocarcinomas) arise from simple epithelial tissue. Additionally, these antigens harbor a wide range of inherent immunogenicity, requiring alterations in formulation and dosage. Due to their homogenous association with invasive disease, and for reasons outlined above, it is very likely these epitopes will be poorly immunogenic compared to other immune modulatory agents. To solve these clinical challenges, patients previously immunized to generate cancer-specific B-cells can be skin tested with various doses of the synthetic or recombinant fully-characterized vaccine components. Due to their previous antigenic exposure, ASI-treated patients will demonstrate various levels of DTH reactions to these compositions when evaluated in a manner analogous to allergen skin testing (Bloemena et al., 1993). Based on these initial tests, the identity of the most potent antigens and required doses can be titrated for the final concentrations utilized in a prophylactic vaccine cocktail. After phase I/II studies have been performed to demonstrate safety and immunogenicity, it should be possible to use this cassette of synthetic or recombinant antigens as a prophylactic vaccine to elicit long-term memory responses in healthy individuals against emergent malignant disease.

Thus, utilizing the presently-defined “outside-in” immunological methods, cancer-specific, ASI-HuMabs are generated for the identification of cancer-specific epitopes, which are then physically recapitulated to create antigens capable of eliciting an immune response against adenocarcinoma development. These ASI-HuMabs are generated from B-cells acquired from patients immunized with autologous, live, tumor cell vaccines. The resulting antibodies are screened and selected to obtain HuMabs with broad specificity for adenocarcinomas, while being substantially non-reactive with normal tissue. In a particular aspect, the autologous, live, tumor cell vaccines are prepared from colon tumors. In another particular aspect, the HuMabs with broad specificity for human adenocarcinomas are selected from the group consisting of 16.88 and MCA 28A32. These HuMabs are then used as molecular probes to identify and characterize the amino acid sequence of the specific epitopes of the corresponding tumor antigens. Specific antigens corresponding to the base sequence of the identified epitopes are then synthesized or recombinantly expressed and screened as discussed herein to provide compositions capable of eliciting an immunogenic response as defined herein.

The following Examples are intended to be illustrative and not limiting.

### Example 1: 88BV59 and Associated Epitope CTA 16.88

The generation of ASI-HuMab 88BV59 is intrinsically linked with patient-specific treatment of cancer and the resulting efficacy of this approach. OncoVAX® is an ASI stimulating a patient's immune system against autologous tumor cells. To prepare OncoVAX®, a patient's own tumor is excised, enzymatically dissociated to separate cells into a single cell suspension, sterilized, and gamma-irradiated to render the cells non-dividing and non-tumorigenic, but still metabolically active (U.S. Pat. No. 7,628,996).

The OncoVAX® treatment protocol consists of four vaccinations injected intradermally. The first two injections consist of live, but non-dividing, tumor cells admixed with fresh-frozen mycobacteria of the TICE Bacillus Calmette-Guerin (BCG) strain. The third and fourth immunizations consist of the remaining live, non-dividing tumor cells, but without BCG. The first three immunizations are delivered weekly approximately 30 days after surgical resection of the primary tumor and the fourth injection is delivered as a booster six months later. BCG is a powerful bacterium proven to stimulate an immune response for the prevention of tuberculosis infection and the treatment of superficial bladder cancer. Consequently, this adjuvant represents a critical component of the vaccination process.

All four doses of OncoVAX® are developed from the tumor obtained after surgical resection of stage II/III colon cancer. In a prospectively randomized and stratified phase III clinical trial (Vermorken et al., 1999), the cell-mediated immune response stimulated by OncoVAX® treatment lead to statistically significant improvements in disease-free survival and overall survival for stage II colon cancer patients (Hanna et al., 2006). This benefit persisted over 15 years of patient follow-up (de Weger et al., 2012), indicating robust immunity with profound immunologic memory.

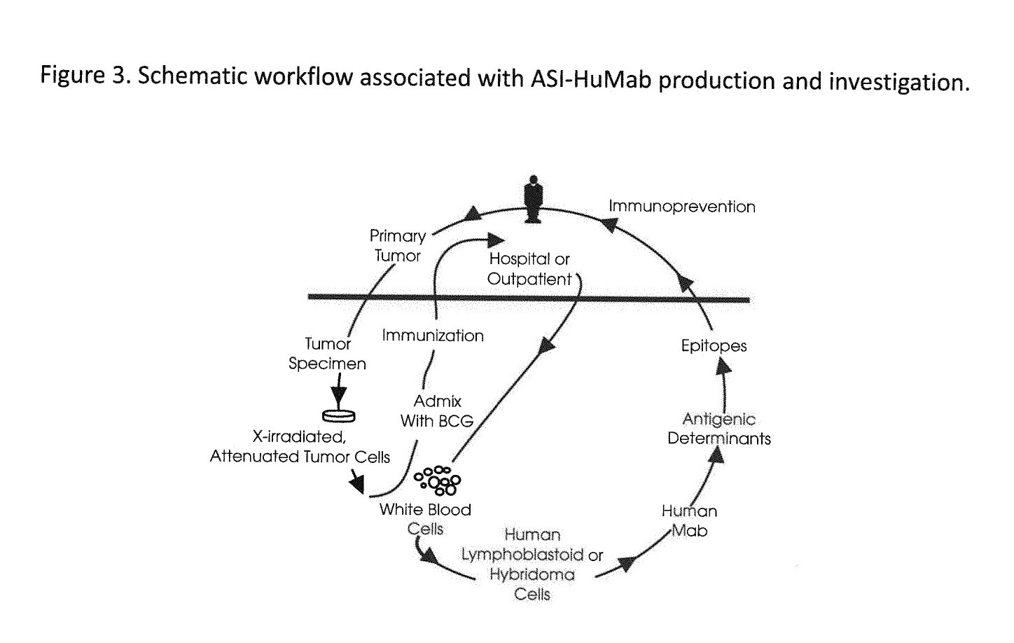

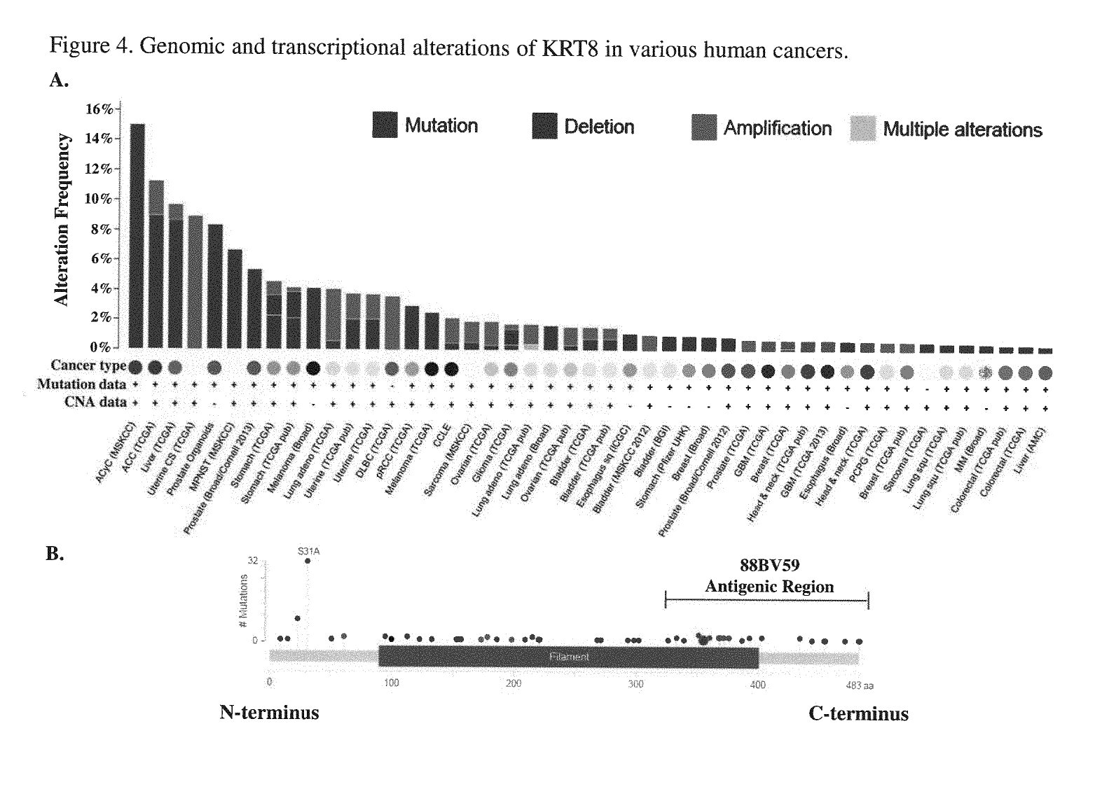

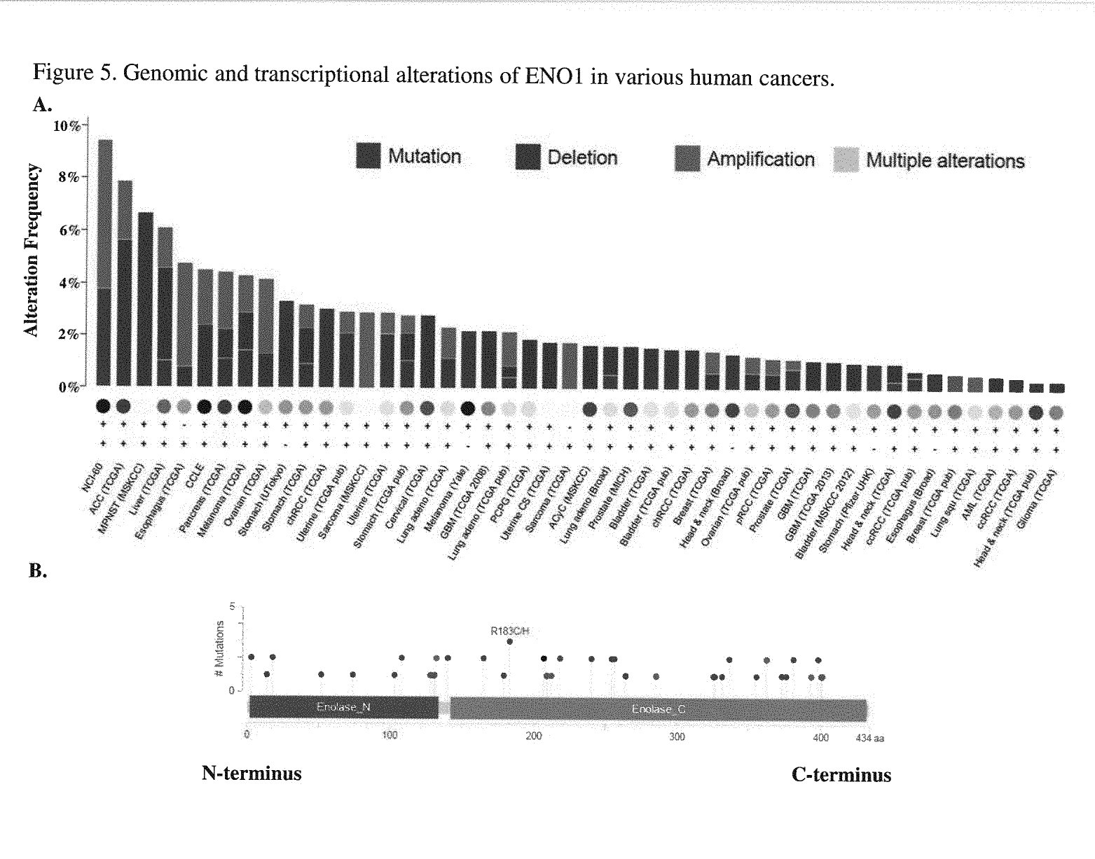

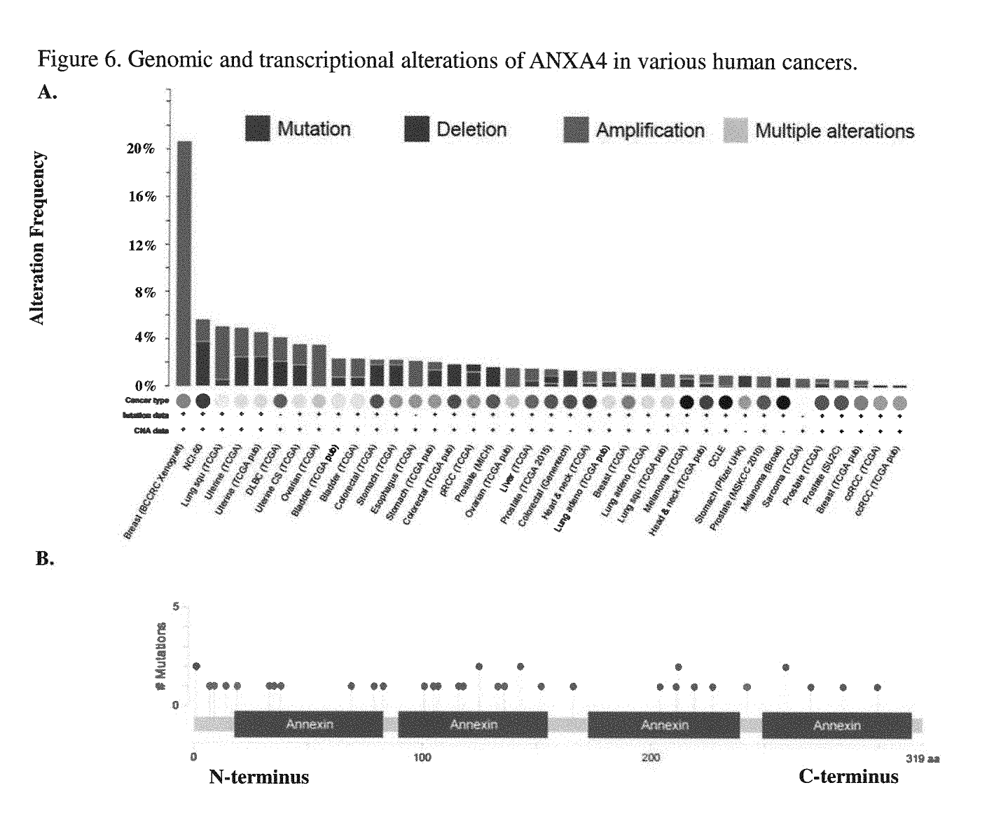

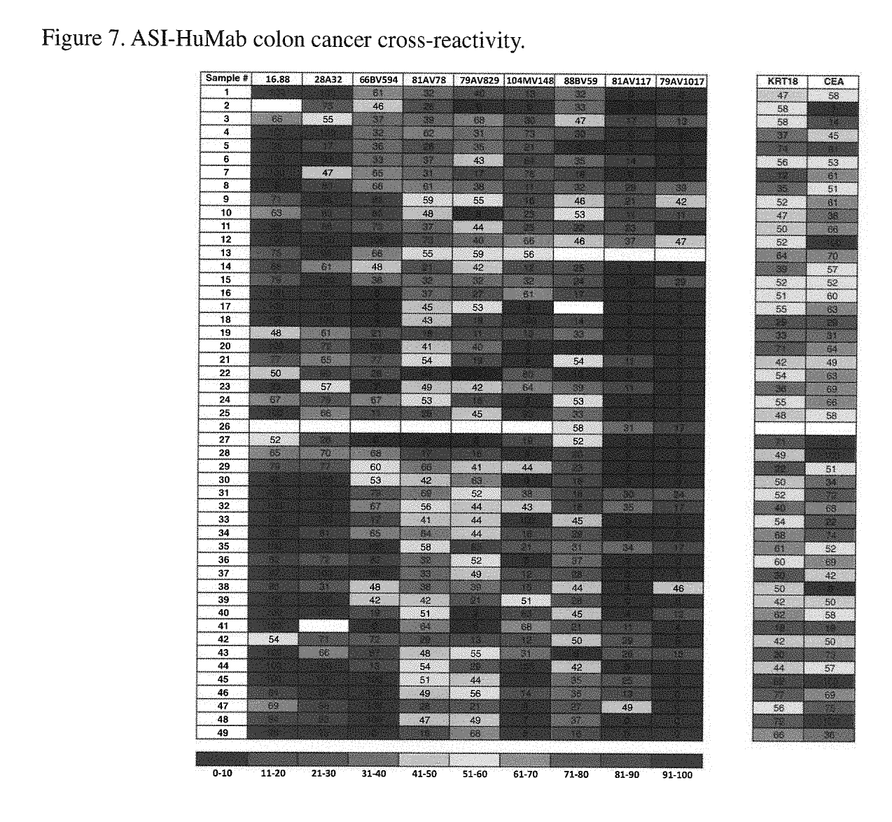

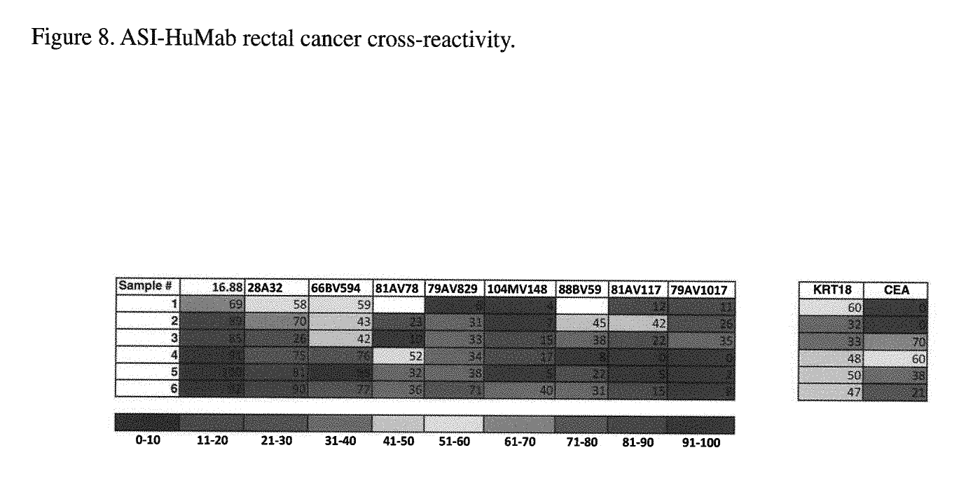

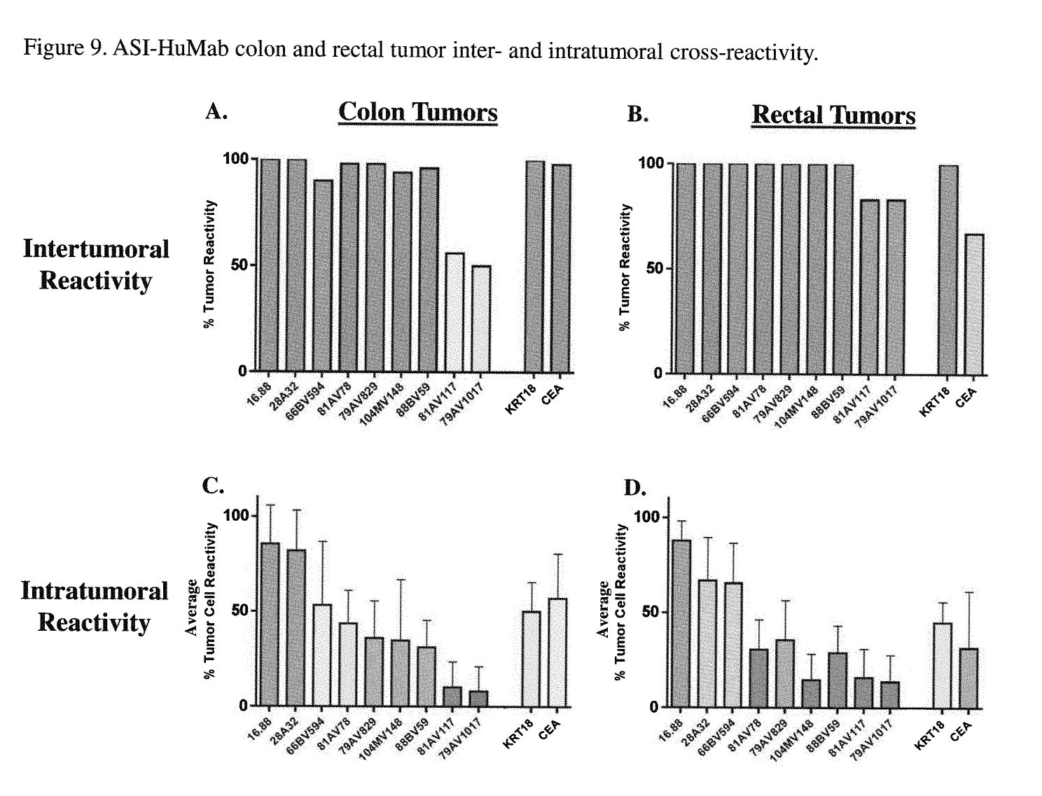

We speculated during the course of immunization, which was developed primarily for the stimulation of a strong cell-mediated immune response, a transient humoral immune response could exist. In several studies, the most productive hybridoma fusions were obtained from B-cells taken 1 week after the first and 1 week after the second immunization (Haspel et al., 1985). No successful fusions with regard to cell-surface antigen-targeting antibodies were obtained in fractions prior to immunization. Thus, by embracing tumor heterogeneity as a source of clinical benefit, a surprising wealth of patient-derived B-cells for cancer-specific HuMab production was discovered among the peripheral blood lymphocytes (FIG. 3).

Approximately 20% of the cultures initially tested produced human immunoglobulin with 15.6% of the HuMabs binding to colon tumor cell antigens (Haspel et al., 1985). Our initial results demonstrated that human colon cancer can be immunogenic if presented to the immune system in the correct manner. Additionally, the antigens recognized are not individually specific, but rather may be expressed on tumors from various patients (FIG. 1). Results with our HuMabs suggested that colorectal tumors appear to express multiple, cross-reactive tumor-associated antigens (Haspel et al, 1985). While no one antibody was able to recognize all tumors in the initial study, a cocktail of two complementary antibodies could achieve the broad reactivity necessary for potential in vivo cancer diagnosis and therapeutic applications (FIG. 2) (Haspel et al, 1985). While therapeutics were the focus of these initial studies and patents, modern techniques and laboratory approaches may be able to utilize this data as the foundation of a novel and intriguing concept: unappreciated tumor homogeneity on the immunologic level may be leveraged for cancer prevention.

Building on these 10 antibodies, a similar HuMab developed later (88BV59) with the same approach was screened in a similar manner (U.S. Pat. No. 5,951,985). However, these initial studies were limited in their scope, quality, and did not recognize the full utility of this agent. Initial immunohistochemical screens were performed against a limited array of cancer cell lines, which poorly recapitulate in vivo disease, and did not adequately represent the diversity of tumor subclasses (i.e. melanoma, lymphoma, etc). Additionally, tumor cell lines often experience genetic drift with continued passage and many develop gene signatures unique from the human disease they purport to represent. These cells also over-represent tumor suppressor loss, a key genetic driver in cancer progression.

Since this initial patent filing, we have screened 88BV59 against a much wider panel of patient-derived tumor samples and normal tissues (Tables 1 and 2). Using biotinylated 88BV59 and an avidin-biotin peroxidase detection system, 88BV59 immunoreactivity was evaluated across a variety of concentrations and diverse tumor subtypes. At the lowest concentration evaluated (0.156 μg/ml) 88BV59 reacted strongly with cells from at least 86% of colon, breast, lung, and stomach adenocarcinomas (Table 1). 88BV59 staining was also evident in 100% of ovarian and prostatic carcinomas.

Most importantly for establishing cancer-specificity, the limited positive staining observed with 88BV59 against a wider panel of normal tissue was largely artifactual (Table 2, see pathology notes) with recognition of the brush borders or weak focal reactivity. The normal tissue which did react to some degree with 88BV59 at these concentrations almost exclusively consisted of glandular structures (i.e. lacrimal gland and breast ductal tissue). With this expanded immunoreactivity profile, it could now be determined that 88BV59 is not a broad “tumor”-specific antibody as previously claimed (U.S. Pat. No. 5,951,985), but rather an “adenocarcinoma”-specific immunoglobulin. Based on these most recent results, this antibody would have little to no utility in the remaining 40% of annual cancer incidence not derived from simple epithelium.

Based on the totality of our most recent findings, it can now be determined that 88BV59 is broadly-specific for cancers which originate from simple epithelium, e.g., cells responsible for creating secretory, glandular structures. This tissue specificity is consistent with the vaccination source material, as these hybridomas were isolated from patients treated with their own adenocarcinomas pursuant to the OncoVAX® process. The current conclusion could be made from this data because the updated cohort includes a more diverse and representative sample set of cancer subclasses. We now know 88BV59 has little to no reactivity with melanoma, lymphoma, or squamous cell carcinomas arising in the head and neck (Table 1). These cancers originate from neural crest, lymphoid, and squamous tissue, respectively, not simple epithelium. This also explains the excellent staining of lung adenocarcinomas with 88BV59, but the moderate to poor staining of lung squamous carcinomas.

It was previously demonstrated the predominant epitope within the 88BV59 antigen (CTA 16.88) is cytokeratin-8 (KRT8) (U.S. Pat. No. 5,951,985), an intermediate filament protein commonly expressed in simple epithelium. This antigen was previously discovered with a similar ASI-HuMab MCA 16.88 (U.S. Pat. No. 5,338,832). A number of 88BV59-specific amino acid sequences within KRT8 were mapped and reported as linear epitopes comprising a more complicated conformational epitope within the tumor-associated antigen CTA 16.88 (U.S. Pat. No. 5,951,985). However, the epitopes claimed were almost exclusively wild-type sequences. If these sequences are the major antigenic determinants of 88BV59, the surprising degree of cancer-specificity observed with this antibody cannot not be explained by epitope sequence alone. If this were true, 88BV59 would also potently recognize KRT8 expression in simple epithelium throughout the body.

One possibility is the conformational epitope recognized by 88BV59 is created by a cancer-specific protein-protein interaction. While KRT8 was noted to be the major antigenic determinant of 88BV59 binding, KRT18 and KRT19 were also identified as components of the CTA 16.88 complex (U.S. Pat. No. 5,951,985). It is quite possible this heterotrimeric complex is not typically represented in normal tissue and this unique protein-protein interaction has immunogenic specificity and perhaps oncogenic function.

To determine the mutational status of KRT8 in cancer, we queried publically available tumor sequencing data (The Cancer Genome Atlas, TCGA) to catalogue known somatic mutations and gene expression alterations of this gene in >21,000 different tumor samples. Across many different types of cancer, including adenocarcinomas, KRT8 mutations are extremely rare (FIG. 4A) (Gao et al., Sci Signal, 6:p11, 2013; Cerami et al., Cancer Discov, 2:401-404, 2012). Additionally, the alterations which do occur are evenly distributed across the protein sequence making a common, mutation-based epitope for antibody recognition very unlikely (FIG. 4B). One mutational hotspot exists for KRT8 (S31A) and this only occurs in 8.9% of hepatocellular carcinomas (HCCs) outside the 88BV59 binding region. At the time of the original 88BV59 studies, widespread tumor sequencing was not available and it could not be determined whether amino acid alterations which were different than the reported reference sequence were truly cancer-specific mutations or simply patient-specific SNP variants.

Further analysis by the present inventors utilizing modern protein databases has determined the previously reported epitope sequences are almost exclusively wild-type, except for Seq. No. 4 (U.S. Pat. No. 5,951,985). This peptide fragment has three alterations: S417G, G429D, and S432D. The first two are previously reported natural variants (SNPs) of KRT8. However, the third is a substitution at a significant functional site within the KRT8 tail region. KRT8 S432 is normally phosphorylated by CaMKII or MAPK in response to epithelial growth factor (EGF) stimulation, an important signaling molecule responsible for stimulating cell growth. Following phosphorylation of S432, subcellular reorganization of the cytokeratin filaments concentrates these complexes at the cell membrane and enhances cell motility. Furthermore, aspartic acid (D) is a well-known phospho-serine mimetic, an amino acid which can mimic the function of phosphorylated serine. Thus, positive recognition of S432D by 88BV59 could implicate another steric requirement for the cancer-specificity of this antibody: EGF-stimulated KRT-8 which translocates the CTA 16.88 complex to the cell membrane for enhanced cell motility and immune recognition.

Taken together, it is highly likely that although KRT8 represents the majority of the 88BV59 antigen, an overall heterotrimeric complex (KRT8/KRT18/KRT19) of wild-type proteins creates a unique conformational epitope which generates the cancer specificity of this antibody. Based on somatic DNA sequencing or gene expression profiling, neither KRT8, KRT18, nor KRT19 would be selected as cancer-specific antigens (FIG. 4); the alterations observed using inside-out screening methods are too sporadic to insinuate any degree of cancer specificity for these proteins. By contrast, because no cancer-specific protein sequences were required to generate this cancer-specific antigen, an ASI-guided “outside-in” approach is the only means of detecting this clinically meaningful, malignant immunome component. The existence of a broadly-specific, adenocarcinoma antigen discovered in this manner should raise concerns about how inside-out approaches may not adequately predict immunological incidence or relevancy.

### Example 2: MCA 28A32 and Epitope

The identification and characterization of ASI-HuMab MCA 28A32 provides another powerful example of the “outside-in” approach to identifying broadly-reactive carcinoma antigenic determinants. MCA 28A32 was produced by the same autologous tumor inoculation methods as 88BV59 and demonstrated broad carcinoma reactivity with minimal recognition of normal tissue (U.S. Pat. No. 5,521,285). The antigen recognized by this antibody (CTAA 28A32) is also a multi-protein complex comprising of 50kD, 46kD, 36kD, and 32kD subunits. Previous studies determined the in vitro T-cell reactive protein was the 46kD subunit, identified by Edman degradation as α-enolase (ENO1). This protein normally functions as a glycolytic enzyme with near ubiquitous expression throughout the body. However, initial investigations determined MCA 28A32 immunohistochemical staining was completely negative for normal breast, stomach, lung, and colon tissue. While the specific MCA 28A32 binding sites have yet to be characterized by amino acid sequence, it is known this antibody physically associated with CTAA 28A32-46kD and -36kD (Pomato et al., 1994), suggesting the −50kD and -32kD subunits co-immunoprecipitate with the complex. A modern reassessment of the sequences used to identify the CTAA 28A32 subunits is instructive for elucidating the power of outside-in antigenic determination.

At the time of the initial investigations, DNA- and protein-based databases were sparse. The peptide fragment associated with the 46kD subunit had 68% homology with the reference sequence for ENO1 (U.S. Pat. No. 5,521,285). It has since been determined this fragment also has 74% identity with c-myc binding protein (MYCBP); however, that molecule has a molecular weight of 12kD and is much too small to represent this protein. The degree of ENO1 variation (32%) observed in this fragment seemed like a reasonable “cancer-specific variant” at the time because large-scale cancer sequencing projects had yet to be undertaken. A modern reassessment of this peptide fragment demonstrates a degree of sequence diversity not evident in current ENO1 cancer mutation analyses (FIG. 5A). In fact, somatic mutations and gene expression alterations of ENO1 are generally not a common occurrence in cancer (Cerami et al., 2012; Gao et al., 2013), let alone to the degree observed in this small fragment of CTAA 28A32-46kD. Furthermore, the somatic mutations which have been observed in ENO1 are evenly distributed across the gene and no obvious hotspots for cancer-specific antibody binding are evident (FIG. 5B) (Cerami et al., 2012; Gao et al., 2013).

Previous studies also identified the 32kD protein as annexin A4 (ANXA4) (Pomato et al., 1994). Annexins are calcium-dependent phospholipid binding proteins which often serve as membrane anchors or mediators of exocytosis. Furthermore, ANXA4 is almost exclusively expressed in epithelial cells, thus its inclusion within an adenocarcinoma antigenic determinant is not unexpected. Similar to ENO1, the specific amino acid sequences of CTAA 28A32-32kD are variants of wild-type ANXA4 (Table 3) (Pomato et al., 1994). Some fragments (peptides 7-10) demonstrate 100% homology with the ANXA4 reference sequence, while others are highly variant (peptide 5, 11, 12). Meanwhile, modern DNA sequencing studies have yet to identify common somatic mutations in ANXA4 (FIG. 6A) and no obvious hotspots are observed (FIG. 6B) (Cerami et al., 2012; Gao et al., 2013). Although, it is interesting to note the expression of ANXA4 was upregulated in at least one breast cancer study (20%), this involved a small cohort (3/15 samples) of patient-derived xenografts. However, other studies have determined that annexin A1, A2, A3, A4, A6, A8, and A11 are upregulated in multiple cancer subtypes.

TABLE 3
Partial protein sequence analysis of CTAA
28A32-32K

| | | ^{a}Percent |
|---|---|---|
| Peptide | Sequence | Identity |
| Peptide 1 | ^{b}ATKGQTVKAASGFNA | 93 |
| Annexin A4 | ATKGGTVKAASGFHA | |
| Peptide 2 | ^{c}LYQFNAMELAQTLXKAMK | 76 |
| Annexin A4 | ASGFNAMEDAQTLRKAMK | |
| Peptide 3 | STIGRDLIDDLKSATPIA | 72 |
| Annexin A4 | STIGRDLIDDLKSELSGN | |
| Peptide 4 | PTVAYDDVQELRRA | 95 |
| Annexin A4 | PTVAY DVQELQRA | |
| Peptide 5 | KHA F KGAFTNELV | 46 |
| Annexin A4 | QRAMKGAGTDEGC | |
| Peptide 6 | EAGTDFGXLIEILASRTPEE | 89 |
| Annexin A4 | GAGTDEGCLIEILASRTPEE | |
| Peptide 7 | SLEYDIR | 100 |
| Annexin A4 | SLEYDIR | |
| Peptide 8 | HLLHVFDEKY | 100 |
| Annexin A4 | HLLHVFDEKY | |
| Peptide 9 | SETSGXFEXA | 100 |
| Annexin A4 | SETSGXFEXA | |
| Peptide 10 | KGLGTDDNTLIRVN | 100 |
| Annexin A4 | KGLGTDDNTLIRVN | |
| Peptide 11 | VSRREHFLRNTRKEFYXNIATSLY | 43 |
| Annexin A4 | VSRAEIDMLDIRAHFKRLYGKSLY | |
| Peptide 12 | IATSLYRQYAXVLL | 23 |
| Annexin A4 | YGKSLYSFIKGDTS | |
| Total Residues | 171 | 74 |
| Sequenced: | | |

^{a}Percentage of amino acids which are identical in CTAA28A32-32K compared to annexin A4
^{b}Underline indicates variation from annexin A4 reference sequence
^{c}“X” indicates amino acid could not be determined at that position

While ENO1 and ANXA4 were previously identified, the protein with an estimated molecular weight of 50kD could not be characterized during the initial investigations. This peptide can now be identified by the present inventors as a cleaved form of glucose-6-phosphate isomerase (GPI). This peptide fragment has strong homology (79%) with GPI and also contains a sugar isomerase (SIS) superfamily conserved domain. This protein normally has a native molecular weight of 63kD and represents the second step in the glycolysis pathway. However, a smaller, cleaved variant of the same protein, termed neuroleukin (56kD), is a secreted form of the enzyme which has neurotrophic and cancer-specific motility functions. While full-length dimeric forms of GPI are enzymatically active, the cell motility functions of neuroleukin are restricted to inactive monomeric forms. Previous studies have also observed isoforms of GPI complexed with other enolase family members.

The totality of these results suggest CTAA 28A32 has cancer-specific expression (antibody cross-reactivity) and function; however, the exact role of CTAA 28A32 in tumor development has yet to be elucidated. A potential function for CTAA 28A32 involves aiding cell motility. Enolases, as well as annexins, have plasminogen binding capabilities when surface expressed on cancer cells. Extracellular localization of these proteins aids plasminogen activation and enhances cancer motility and invasion through the extracellular matrix. The steric requirement of ANX4A acting as an extracellular membrane anchor may also explain why CTAA 28A32-32kD is not sufficiently recognized by MCA 28A32, as it is buried between the surface exposed subunits and the plasma membrane, not available for immunoglobulin recognition. Based on these observations and due to the extensive amino acid sequence variations observed, it is likely these enzymes are not active in their classical roles, but aid cell motility in a cancer-specific heterotetrameric complex expressed on the cell surface rather than serving to provide enhanced intracellular glycolytic activity.

Despite advances in modern protein databases, the present inventors still cannot determined the identity of CTAA 28A32-36kD. Thus, the degree of amino acid variation observed in the sequenced fragments as well as their co-association may be completely novel. Furthermore, the current data suggests no single aspect of CTAA 28A32 (i.e. homogeneous immunohistochemical recognition of colon tumors, highly variant peptide sequences, unique tetrameric complex) could have been anticipated or discovered using “inside-out” approaches.

### Example 3: Determining Intertumoral vs. Intratumoral ASI-HuMab Reactivity

When 88BV59 was first developed and patented (U.S. Pat. No. 5,951,985), its degree of intertumoral cross-reactivity was immediately appreciated for the ability to create novel cancer therapeutics or diagnostics. In fact, 88BV59 was later conjugated with technetium (^{99m}Tc) to create votumumab (HumaSPECT®), an antibody used to visualize and clinically monitor cancer recurrence. This agent ultimately received approval by the European Medicines Agency (EMA) for monitoring recurrent and/or metastatic colorectal carcinomas. In a phase III multicenter trial, votumumab was more accurate than computed tomography (CT) for predicting non-resectability of disease (60% vs. 29%, p<0.001) (Wolff et al., 1998). A follow-up study determined votumumab had improved sensitivity and specificity over CT in determining the presence and location of recurrent and/or metastatic colorectal cancer (Serafini et al., 1998). Consequently, immunohistochemical (Table 1) and clinical trial results both confirm the intertumoral cross-reactivity and specificity of 88BV59. However, to create an efficacious preventive vaccine, the antigenic determinants used must have significant inter- and intratumoral representation. Therefore, the ASI-HuMabs used to guide this characterization must also demonstrate this type of reactivity across and within many tumor samples.

To determine whether antigenic determinants which satisfy both of these conditions exist, dissociated tumor preparations used for OncoVAX® treatment were screened against a panel of 9 ASI-HuMabs. These screens included 49 colon tumors (FIG. 7) and 6 rectal tumors (FIG. 8). The heat maps provided demonstrate the percentage of positive tumor cells within a given tumor preparation as determined by immunohistochemistry. Cytokeratin 18 (KRT18) and carcinoembryonic antigen (CEA) staining is also provided as a benchmark for common colon adenocarcinoma histochemical markers. KRT18 is a common marker for adenocarcinomas; however, this protein is also highly expressed in simple epithelium, and thus displays poor cancer specificity. Circulating CEA is currently used as a plasma marker for monitoring colon cancer recurrence; however, CEA can be expressed at low levels in normal tissue and efforts to leverage this protein as an immunotherapeutic have failed.

Using a screen of this design, one can establish the intertumoral (FIG. 9A,B) and intratumoral (FIG. 9C,D) reactivity of each ASI-HuMab. The first observation is ASI-HuMabs commonly demonstrate a high degree of intertumoral reactivity. KRT18 and CEA also recognize nearly all colon adenocarcinomas (FIG. 9A), but they do not possess the unique specificity for cancer vs. normal tissue these antibodies exhibit. As mentioned above, this degree of intertumoral cross-reactivity with minimal normal recognition has previously been clinically confirmed for 88BV59 (Wolff et al., 1998; Serafini et al., 1998). However, only two ASI-HuMabs (16.88 and 28A32) demonstrate consistent inter- and intratumoral reactivity in a large cohort of colon tumors and a small cohort of rectal tumors. These results confirm that homogenous adenocarcinoma antigenic determinants do exist and they can be recognized and later characterized using ASI-HuMabs as molecular probes.

One surprising result was the relatively poor intratumoral reactivity of 88BV59 (average percent reactivity in colon tumors: 31±14%). This is especially interesting given 16.88 and 88BV59 recognize the same antigen (CTAA 16.88), albeit via different epitopes. We believe these results suggest 16.88 recognizes a structural, conformational epitope required for KRT8/18/19 heterotrimeric association, whereas the epitope for 88BV59 may be more transient. If 88BV59 recognition requires a site-specific phosphorylation, as postulated above (S432D, Sequence 4, U.S. Pat. No. 5,951,985), it is possible this epitope is more transient than 16.88, as phosphorylations are highly reversible, dynamic post-translational modifications. Thus, while the existence of the 88BV59 epitope is clearly cancer-specific compared to normal tissue, it may exist transiently within tumors. Yet, this transient occurrence may occur often enough to be relatively common across multiple tumors for diagnostic use.

Based on the current cross-reactivity results, a physical recapitulation of CTAA 16.88, 28A32, and to a lesser degree 88BV59, could represent potential components of an adenocarcinoma preventive vaccine. However, while humoral detection is an important component to cancer prevention, T-cell-mediated events must also occur if early tumors are to be recognized and destroyed. Thus, each antigen must be tested for an ability to elicit a T-cell response.

### Example 4: Establishing Antigenic Determinant T-Cell Reactivity

To determine if either antigen (CTAA 16.88 or 28A32) can stimulate a significant in vivo immune response, components of both targets were evaluated by skin testing in patients being treated for colon cancer with ASI (Bloemena et al., 1993). By definition, because these patients were the source of the ASI-HuMabs and these antigenic determinants are a common occurrence, these patients are primed to inform investigators which antigens demonstrate T-cell reactivity. Consequently, MCAs 16.88 and 28A32 were used to immunoprecipitate cognate antigens from a colon cancer cell line (HT-29) and the resulting products were purified using HPLC.

Purified CTAA 16.88 and various components of CTAA 28A32 (−50kD, −46kD, −36kD, −32kD) were injected intradermally (0.1 mg/100 μl PBS) in ten patients receiving OncoVAX® ASI vaccination (Table 4) (Bloemena et al., 1993). No patients reacted positively with CTAA 16.88. Seven patients experienced erythromatous reactions against a combination of CTAA 28A32-50kD and -32kD. Immunohistochemical analysis determined nearly all primary tumor sections from these patients reacted positively with MCA 16.88, MCA 28A32, and rabbit sera from animals inoculated with CTAA 28A32-32kD. Thus, lack of in vivo reactivity is not due to the antigen being absent from the original vaccination material.

TABLE 4
Comparison of antigen expression by immunohistochemical
analysis with DTH response

| | | | CTAA | | |
|---|---|---|---|---|---|
| | CTAA 16.88 | | 28A32-50 kD | | Rabbit |
| | DTH | MCA | and -32 kD | MCA | anti-CTAA |
| Patient | response | 16.88 | DTH response | 28A32 | 28A32-32 kD |
| 1 | —^{a} | 1+^{b} | 10^{a} | 1+^{b} | 4+^{b} |
| 2 | — | 1+ | 18 | 1+ | 1+ |
| 3 | — | 3+ | 9 | 3+ | 3+ |
| 4 | — | — | 12 | 2+ | 3+ |
| 5 | — | 2+ | — | 2+ | — |
| 6 | — | 3+ | — | 4+ | 2+ |
| 7 | — | 2+ | 6 | 2+ | 4+ |
| 8 | — | 4+ | 8 | 4+ | 3+ |
| 9 | — | 3+ | 10 | 3+ | 3+ |
| 10 | — | — | — | 1+ | 2+ |

^{a}Mean diameter of erythema (mm). No response (—).
^{b}Staining intensity: (—) negative to very weak focal staining (<5%); 1+, weak staining; 2+, weak to moderate staining; 3+, moderate staining; 4+, strong staining.

This study also provided critical evidence that although many tumor-specific protein complexes and antigens may exist, not all provide T-cell reactive epitopes. While epitopes associated with CTAA 16.88 may be adequate targets for cancer-specific diagnostics or radiological treatments, T-cell reactive epitopes will be the most useful for the generation of a prophylactic vaccine. Ideally, these antigens should be capable of simultaneously eliciting a humoral response for B-cell recognition and a cytotoxic T-cell response capable of destroying cells presenting carcinoma-specific epitopes.

These previous studies also serve as an excellent reminder that peptide synthesis and recombinant protein technologies were exceedingly difficult during the initial evaluation of these antibodies. Large-scale production of antibody epitopes could not have been anticipated or attempted at this time. Industrial-scale immunoprecipitation would not have been a feasible approach for producing a homogeneous adenocarcinoma antigenic determinant vaccine. At the time, all antigens had to be immunoprecipitated and purified from cancer cell lines, greatly limiting the amount of material which could be used. No adjuvants were used in these initial studies and higher protein concentrations (>0.1 mg) are likely required to stimulate a potent immune response against targets which are predefined as poorly immunogenic.

The most intriguing result associated with CTAA 28A32 skin reactions is the relatively common immune recognition of the 32kD component. When anti-rabbit sera was raised against each purified component of 28A32, only the 32kD serum was able to specifically react with tumor sections during immunohistochemical analysis (Bloemena et al., 1993). Additionally, previous reports in OncoVAX® patients have demonstrated CTAA 28A32-32kD is the only 28A32 subunit which significantly increases in peripheral T-cell reactivity with OncoVAX® treatment (Table 5) (Ransom et al 1993).

TABLE 5
Proliferative responses to tumor-associated antigens by peripheral blood lymphocytes
autologous tumor-cell BCG immunized patients.

| | Number with positive SI/total tested¹ | | | | | | | | |
|---|---|---|---|---|---|---|---|---|---|
| | | | | After 3^{rd} | | | After 4^{th} | | |
| Antigen | Baseline | | | vaccination | | | vaccination | | |
| CTAA 16.88 | 0/10 | | | 0/11 | | | 0/9 | | |
| CTAA 28A32-50 kD | 0/12 | | | 1/19 | (3.0) | 5% | 0/11 | | |
| CTAA 28A32-46 kD | 0/12 | | | 1/18 | (3.3) | 6% | 1/11 | (9.1) | 9% |
| CTAA 28A32-36 kD | 0/12 | | | 0/18 | | | 2/10 | (4.5-4.8) | 20% |
| CTAA 28A32-32 kD | 0/12 | | | 3/20 | (3.3-9.5) | 15% | 7/13 | (3.0-4.8) | 54% |
| PPD | 3/13 | (10-108) | 23% | 12/24 | (13-330) | 50% | 9/11 | (5-45) | 82% |

^{a}SI (given in parentheses) ≧3.0 were considered positive.

During the development of the OncoVAX® treatment protocol, the addition of the fourth vaccination (booster) was a critical turning point for consistent and clinically meaningful prevention of tumor recurrence (Vermorken et al., 1999). We believe the enhanced immunological recognition of CTAA 28A32-32kD between the third and fourth vaccination is a molecular fingerprint highlighting the importance of this target in mediating effective tumor recognition and long-term surveillance against recurrent disease. Due to its near ubiquitous expression (humoral reactivity) in colon cancer samples and immunological reactivity (cell-mediated reactivity), this epitope would constitute a significant component of an adenocarcinoma prophylactic vaccine. Expanded screening and T-cell reactivity studies with previous and future ASI-HuMabs will further elucidate adenocarcinoma antigenic determinants with homogenous inter- and intratumoral adenocarcinoma expression, limited normal tissue recognition, and peripheral T-cell reactivity in ASI-treated patients.

### CONCLUSION

In all cases, MCAs 16.88, 28A32, and 88BV59 were generated by challenging human immune systems with an outside-in approach for tumor-specific antigen discovery. All three antibodies immunoprecipitate multi-subunit complexes which demonstrate carcinoma specificity. In the first case, a cancer-specific complex of largely wild-type proteins (CTAA 16.88), may confer adenocarcinoma reactivity. However, the relatively normal amino acid sequences or reliance on transient post-translational modifications may limit T-cell reactivity. In the second case (CTAA 28A32), previously undetected and unappreciated protein variants and complexes may be responsible for tumor specificity and novel cancer-specific functionality. Highly unique protein sequences within this antigen may also create attractive humoral and cell-based targets for future epitope-based vaccine development.

With these antibodies and the newly added context of modern genetic and proteomic analysis, we have a number of examples of how inter- and intratumoral immunoglobulin cross-reactivity can be discovered without prior evidence of predicted cancer-specificity with “inside-out” profiling studies. Furthermore, the antibody- and T-cell-based screens described herein have identified CTAA 28A32 as an adenocarcinoma antigenic determinant which could constitute at least one significant component of an anti-cancer prophylactic vaccine. Other antibodies with impressive intertumoral, but less significant intratumoral reactivity (i.e. 88BV59), may be required as components of a final composition in order to address subclone populations not targeted by antigenic determinants with excellent intratumoral representation. In either instance, too many alterations, subcellular localizations, and conjugations are critical for tumor homeostasis which are not evident on the genetic level. While not wishing to be bound by theory, it appears that due to the constraints of the tools previously used to characterize tumors, a high level of genetic heterogeneity was identified while a surprising level of antigenic homogeneity was unappreciated.

Although the embodiments have been described with reference to the Tables, Figures, and specific examples, it will be readily appreciated by those skilled in the art that many modifications and adaptations of the compositions and methods described herein are possible without departure from the spirit and scope of the embodiments as claimed hereinafter. Thus, it is to be understood that this description is made only by way of example and not as a limitation on the scope of the embodiments as claimed below.

What is claimed is:

1. A method for making a peptide capable of eliciting an immunogenic response to adenocarcinoma tumor cells, comprising;
immunizing a human cancer patient with an autologous live tumor cell vaccine containing live tumor cells obtained from adenocarcinoma source material excised from the patient;
collecting B-cells from peripheral blood lymphocytes of the immunized patient;
isolating human monoclonal antibodies from the B-cells;
screening the antibodies against adenocarcinoma cells from a range of different sources and against normal non-carcinoma cells from similar sources;
identifying antibodies that specifically bind to adenocarcinoma cells;
selecting an antibody having reactivity with a range of adenocarcinoma cells and substantially no reactivity with normal non-carcinoma cells;
identifying a binding site associated with the antibody on the adenocarcinoma cells;
characterizing an epitope from the identified binding site;
preparing a peptide corresponding to the epitope; and
determining if the peptide is capable of eliciting an immune response by a positive delayed hypersensitivity skin test in an immunized patient.

2. The method of claim 1, wherein the human monoclonal antibody is MCA 88BV59.

3. The method of claim 2, wherein the epitope is CTA 88BV59.

4. The method of claim 1, wherein the human monoclonal antibody is MCA 16.88.

5. The method of claim 4, wherein the epitope is CTA 16.88.

6. The method of claim 1, wherein the human monoclonal antibody is MCA 28A32.

7. The method of claim 6, wherein the epitope is CTA 28A32.

8. The method of claim 1, wherein the selected antibody reacts with a wide range of adenocarcinoma cells from different sources.

9. The method of claim 1, wherein the peptide is prepared by recombinant protein expression.

10. The method of claim 1, wherein the peptide is prepared by immunoprecipitation.

11. The method of claim 1, wherein the peptide is prepared by peptide synthesis.

12. An immunogenic composition comprising at least one peptide prepared by the method of claim 1.

13. The immunogenic composition of claim 12, wherein the at least one peptide is a conformational, non-linear adenocarcinoma tumor antigen.

14. The immunogenic composition of claim 12, wherein the at least one peptide characterizes an epitope that occurs in at least 60% of adenocarcinoma tumors.
